(19) United States
(12) Patent Application Publication
(10) Pub. No.: US 2021/0205666 A1
(43) Pub. Date: Jul. 8, 2021

(54) INFORMATION DISTRIBUTION SYSTEM, INFORMATION DISTRIBUTION METHOD, AND PROGRAM RECORDING MEDIUM

(71) Applicant: NEC Corporation, Minato-ku, Tokyo (JP)

(72) Inventors: Kenichiro FUKUSHI, Tokyo (JP); Hiroshi KAJITANI, Tokyo (JP); Chenhui HUANG, Tokyo (JP); Kentaro NAKAHARA, Tokyo (JP)

(73) Assignee: NEC Corporation, Minato-ku, Tokyo (JP)

(21) Appl. No.: 17/269,378

(22) PCT Filed: Aug. 21, 2018
(86) PCT No.: PCT/JP2018/030760

§ 371 Date: Feb. 18, 2021

(51) Int. Cl. A63B 24/00; G16H 50/50; G16H 50/30; G16H 50/70; G16H 40/67; A61B 5/397; A61B 5/103; A61B 5/00

(52) U.S. Cl. CPC A63B 24/0062; G16H 50/50; G16H 50/30; G16H 50/70; A63B 2071/065; A61B 5/397; A61B 5/1038; A61B 5/7278; A61B 5/4866; G16H 40/67

(57) ABSTRACT

In order to distribute content suitable for a user according to a fatigue degree of the user who uses an information distribution service at an appropriate timing, an information distribution device includes a measurement data acquisition means that acquires measurement data regarding a motion of the user, a measurement data analysis unit that analyzes the measurement data acquired by the measurement data acquisition means and calculates a fatigue indicator indicating a degree of fatigue of the user, a content generation unit that generates content on the basis of the fatigue indicator calculated by the measurement data analysis unit, and a notification management unit that distributes the content generated by the content generation unit on the basis of the fatigue indicator.

### TECHNICAL FIELD

The disclosure relates to an information distribution system, an information distribution method, and a program for distributing content.

### BACKGROUND ART

A technology that generates content suitable for a user of an information distribution service and distributes the content to the user at an appropriate timing is required.

PTL 1 discloses an information distribution system that distributes information that matches a request of a user. The system in PTL 1 collects regional information including environmental information and map information, collects distribution target conditions and distribution information from an information provider, and collects personal information from a user terminal. The system in PTL 1 collects an action history including position information regarding the user from the user terminal and analyzes an action pattern of the user on the basis of the collected action history. The information distribution system in PTL 1 collects the position information of the user from the user terminal and distributes distribution information that is searched on the basis of the environmental information, the action pattern, the personal information, and the distribution target condition of the user to the user.

PTL 2 discloses an information processing apparatus that provides content related to position information of a terminal used by a user. The device in PTL 2 specifies a position of the terminal, transmits the content to the terminal in a case where the specified position is included in a distribution region associated with the content, and changes the distribution region in a case where a change condition is satisfied.

PTL 3 discloses a walking assisting device that is attached to a leg of a user and assists walking of the user by applying an assistance force. The walking assisting device in PTL 3 includes a mounting portion that fixes a support portion to the leg of the user, an actuator that applies the assistance force by driving the support portion, and a control unit that drives the actuator. The control unit of the walking assisting device in PTL 3 executes processing for calculating a fatigue degree on the basis of a usage state of the walking assisting device by the user and a physical feature amount of the user and driving the actuator according to an assistance amount determined on the basis of the fatigue degree.

PTL 4 discloses a walking state measurement device that includes ground reaction force estimation means estimating a ground reaction force received by a sole of a pedestrian who is walking from the floor and walking state calculation means calculating a walking state of the pedestrian. The ground reaction force estimation means estimates the ground reaction force using at least one of motion data and lower limb vertical load data of the pedestrian. The walking state calculation means calculates the walking state of the pedestrian using at least one of the motion data and the ground reaction force data.

### CITATION LIST

### Patent Literature

[PTL 1] JP 2002-342367 A
[PTL 2] JP 2017-199122 A
[PTL 3] JP 2012-205621 A
[PTL 4] WO 2018/101071 A

### SUMMARY OF INVENTION

### Technical Problem

According to the technologies of PTLs 1 and 2, the content suitable for the user can be generated on the basis of the action history and the position of the user. However, the technologies do not consider the fatigue degree of the user. Therefore, there has been a problem in that it is not possible to generate the content suitable for the user at an appropriate timing and convenience of a service is deteriorated.

According to the device in PTL 3, the fatigue degree of the user is calculated according to the physical feature amount of the user and the usage state of the walking assisting device and the assistance amount is changed according to the calculated fatigue degree in such a way that it is possible to assist walking according to the fatigue of the user that changes as the user walks.

When the technologies of PTLs 1 to 3 are combined, the content suitable for the user may be generated at an appropriate timing according to the fatigue degree of the user. However, regarding the device in PTL 3, because the user needs to wear the walking assisting device, it is difficult to apply the device to daily life. Therefore, even if the technologies in PTLs 1 to 3 are combined, there has been a problem in that, in a situation in which the user does not wear the walking assisting device, it is not possible to generate the content suitable for the user at an appropriate timing.

An object of the disclosure is to solve the above problems and to provide an information distribution system that can distribute content suitable for a user at an appropriate timing according to a fatigue degree of the user who uses an information distribution service.

### Solution to Problem

An information distribution device according to one aspect of the disclosure includes a measurement data acquisition means that acquires measurement data regarding a motion of a user, a measurement data analysis means that analyzes the measurement data acquired by the measurement data acquisition means and calculates a fatigue indicator that indicates a degree of fatigue of the user, a content generation means that generates content based on the fatigue indicator calculated by the measurement data analysis means, and a notification management means that distributes the content generated by the content generation means on the basis of the fatigue indicator.

An information distribution method according to one aspect of the disclosure includes, by an information processing server, acquiring measurement data regarding a motion of a user, analyzing the acquired measurement data and calculating a fatigue indicator that indicates a degree of fatigue of the user, generating content based on the calculated fatigue indicator, and distributing the generated content on the basis of the fatigue indicator.

A program according to one aspect of the disclosure causes a computer to execute processing including processing for acquiring measurement data regarding a motion of a user, processing for analyzing the acquired measurement data and calculating a fatigue indicator that indicates a degree of fatigue of the user, processing for generating content based on the calculated fatigue indicator, and processing for distributing the generated content on the basis of the fatigue indicator.

### Advantageous Effects of Invention

According to the disclosure, it is possible to provide an information distribution system that can distribute content suitable for a user at an appropriate timing according to a fatigue degree of the user who uses an information distribution service.

### BRIEF DESCRIPTION OF DRAWINGS

FIG. 1 is a block diagram illustrating an example of a configuration of an information distribution system according to a first example embodiment of the disclosure.

FIG. 2 is a block diagram illustrating another example of the configuration of the information distribution system according to the first example embodiment of the disclosure.

FIG. 3 is a block diagram illustrating an example of a configuration of a data acquisition device included in the information distribution system according to the first example embodiment of the disclosure.

FIG. 4 is a block diagram illustrating an example of a configuration of an information distribution server included in the information distribution system according to the first example embodiment of the disclosure.

FIG. 5 is a flowchart for explaining a method for calculating a recovery speed used by the information distribution system according to the first example embodiment of the disclosure.

FIG. 6 is a graph for explaining a method for calculating the recovery speed used by the information distribution system according to the first example embodiment of the disclosure.

FIG. 7 is a block diagram illustrating an example of a configuration of a terminal device included in the information distribution system according to the first example embodiment of the disclosure.

FIG. 8 is a flowchart for explaining an operation of the information distribution system according to the first example embodiment of the disclosure.

FIG. 9 is a block diagram illustrating an example of a configuration of an information distribution system according to a second example embodiment of the disclosure.

FIG. 10 is a block diagram illustrating an example of first content stored in a content database of the information distribution system according to the second example embodiment of the disclosure.

FIG. 11 is a block diagram illustrating an example of a fatigue indicator stored in the content database of the information distribution system according to the second example embodiment of the disclosure.

FIG. 12 is a block diagram illustrating an example of a configuration of a terminal device included in an information distribution system according to a third example embodiment of the disclosure.

FIG. 13 is a block diagram illustrating an example of second content stored in a content database of the information distribution system according to the third example embodiment of the disclosure.

FIG. 14 is a conceptual diagram for explaining an application example of the information distribution system according to the third example embodiment of the disclosure.

FIG. 15 is a flowchart for explaining an operation of the information distribution system according to the third example embodiment of the disclosure.

FIG. 16 is a block diagram illustrating an example of third content stored in a content database of an information distribution system according to a fourth example embodiment of the disclosure.

FIG. 17 is a conceptual diagram for explaining an application example of the information distribution system according to the fourth example embodiment of the disclosure.

FIG. 18 is a flowchart for explaining an operation of the information distribution system according to the fourth example embodiment of the disclosure.

FIG. 19 is a block diagram illustrating an example of a hardware configuration for implementing the information distribution server of the information distribution system according to each example embodiment of the disclosure.

### EXAMPLE EMBODIMENT

Hereinafter, example embodiments of the disclosure will be described with reference to the drawings. However, although technically preferred limitation in order to implement the disclosure is applied to the example embodiments described later, the scope of the invention is not limited as follows. In all the drawings used for description of the example embodiments below, the similar portion is denoted with the same reference numerals unless there are no particular reasons. In the following example embodiments, repetitive description on similar configurations and operations may be omitted. A direction of an arrow in the drawings indicates an example and does not limit a direction of a signal between blocks.

### First Example Embodiment

Next, an information distribution system according to a first example embodiment of the disclosure will be described with reference to the drawings. The information distribution system according to the present example embodiment acquires measurement data for analyzing a fatigue degree of a user from a terminal device used by the user who uses an information distribution service provided by this system. The information distribution system according to the present example embodiment generates content according to an analysis result of the acquired measurement data and distributes the generated content to the terminal device of the user.

(Configuration)

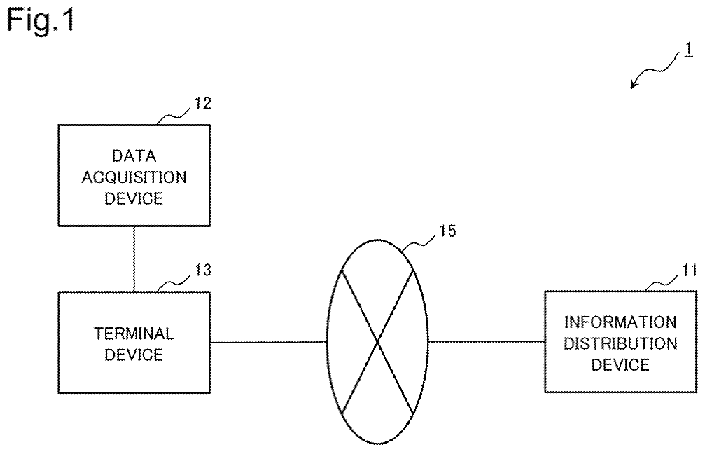

FIG. 1 is a block diagram illustrating an outline of a configuration of an information distribution system 1 according to the present example embodiment. As illustrated in FIG. 1, the information distribution system 1 includes an information distribution device 11, a data acquisition device 12, and a terminal device 13. Although the components of the information distribution system 1 perform data communication with each other, a communication method between the components is not particularly limited. The components of the information distribution system 1 may be wirelessly or wiredly connected to each other.

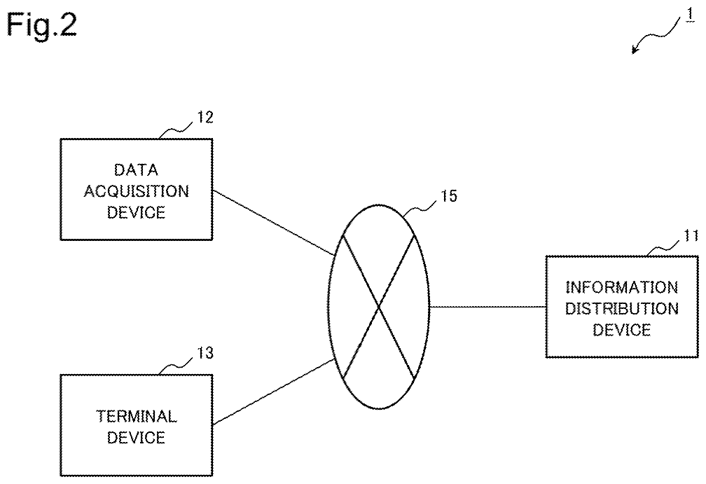

The information distribution device 11 is connected to the terminal device 13 via a network 15. The information distribution device 11 is connected to the data acquisition device 12 via the terminal device 13. As illustrated in FIG. 2, the information distribution device 11 may be configured to be connected to the data acquisition device 12 via the network 15, without via the terminal device 13. The network 15 is a communication network such as the Internet or an intranet. A form of the network 15 is not particularly limited. In a case where the network 15 is implemented as a local network, the network 15 may be added to the configuration of the information distribution system.

For example, the information distribution device 11 can be implemented by a general server device configured in a data center or a cloud. The information distribution device 11 may be implemented by a dedicated server device or a computer that is configured to easily achieve functions of the present example embodiment.

The information distribution device 11 performs data communication with the terminal device 13 used by a user of an information distribution service provided by the information distribution system 1 via the network 15. The information distribution device 11 receives measurement data transmitted from one of the data acquisition device 12 or the terminal device 13. The information distribution device 11 analyzes the received measurement data and generates content according to the analysis result. The information distribution device 11 distributes the generated content to the terminal device 13.

The measurement data analyzed by the information distribution device 11 is motion data and ground reaction force data when a user walks. For example, the information distribution device 11 generates the motion data and the ground reaction force data using detection data detected by an inertial measurement unit (IMU) attached to the user's thigh, knee, top of the foot, or the like. The information distribution device 11 may be configured to estimate the ground reaction force data using the motion data of the user. Furthermore, data other than the motion data and the ground reaction force data may be added to the measurement data.

For example, if software that is called OpenSim (Stanford University) and performs musculo-skeletal model analysis is used, it is possible to calculate time-series data of a muscle activity amount for each predetermined muscle of the user by using the motion data and the ground reaction force of the user. The muscle activity amount is a ratio of a muscle potential that is actually measured from the muscle with respect to a muscle potential measured when the target muscle exerts the maximum power. From the muscle activity amount for each predetermined muscle of the user, it is possible to calculate a metabolic cost of each muscle. In the present example embodiment, the fatigue indicator is calculated using time-series data of the metabolic cost calculated for each muscle, and the content generated on the basis of the fatigue indicator is transmitted to the terminal device 13 of the user. A specific method for calculating the fatigue indicator will be described later. The fatigue indicator may be calculated using the measurement data measured by an activity meter, not the motion data and the ground reaction force data.

The data acquisition device 12 measures the motion data and the ground reaction force data of the user. The data acquisition device 12 transmits the acquired motion data and ground reaction force data to the information distribution device 11 as measurement data.

For example, the data acquisition device 12 is implemented as an IMU attached to a user. In the data acquisition device 12, a device that measures the motion data and a device that measures a ground reaction force may be separately configured, and the data acquisition device 12 may be configured as a single device that measures the motion data and the ground reaction. The data acquisition device 12 may be configured as software or an application installed to the terminal device 13. In that case, it is sufficient that a sensor that measures the motion data and a sensor that measures the ground reaction force be connected to the terminal device 13 and the motion data and the ground reaction be generated by the software or the application installed to the terminal device 13.

The data acquisition device 12 may be configured to estimate the ground reaction force using the motion data based on detection data that is actually measured. For example, based on the method disclosed in WO 2018/101071 A, the ground reaction force can be estimated by using lower limb vertical load data that is measured by using a sensor using an entire sole as a pressure-sensitive surface and motion data measured by a motion measurement device. In the present example embodiment, it is assumed that the lower limb vertical load data be included in the motion data.

The terminal device 13 is connected to the information distribution device 11 via the network 15. In a case of the configuration in FIG. 1, the terminal device 13 is connected to the data acquisition device 12 by wireless communication or wired communication. In a case of the configuration in FIG. 2, it is not necessary to connect the terminal device 13 and the data acquisition device 12.

For example, the terminal device 13 can be implemented by an electronic device that has at least a communication function and a display function. For example, the terminal device 13 can be implemented by a general smartphone, mobile phone, tablet-type personal computer, notebook personal computer, mobile information terminal, game machine, or the like.

The outline of the configuration of the information distribution system 1 has been described above. Next, detailed configurations of the information distribution device 11, the data acquisition device 12, and the terminal device 13 included in the information distribution system 1 will be described with reference to the drawings.

[Data Acquisition Device]

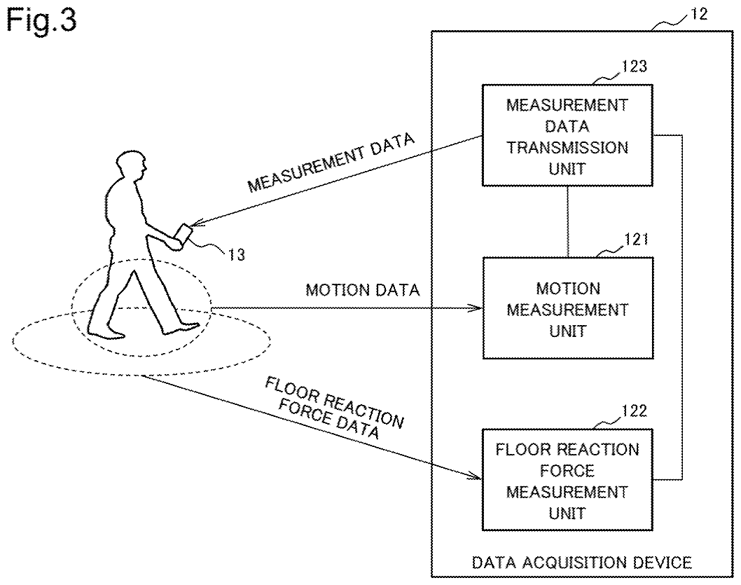

FIG. 3 is a block diagram illustrating an example of the detailed configuration of the data acquisition device 12. As illustrated in FIG. 3, the data acquisition device 12 includes a motion measurement unit 121, a ground reaction force measurement unit 122, and a measurement data transmission unit 123.

The motion measurement unit 121 measures a motion of a user. For example, the motion is information regarding an angle and an angular velocity of each joint, a posture, a position, an acceleration, and an angular velocity of each body segment, or the like when a user's body is assumed as a rigid link model. The motion measurement unit 121 outputs motion data that is a measurement result to the measurement data transmission unit 123.

For example, if at least one of an inertial sensor system, an optical system, a mechanical system, a magnetic system, a video analysis system, or the like is used, the motion of the user can be measured. For example, if an inertial measurement unit (hereinafter, referred to as IMU) including an accelerometer and an angular velocity meter is used, the motion can be measured without using a large device. In that case, if the plurality of IMUs is attached to each portion such as thighs, sural regions, the top of the foot, or the like of both legs of the user, the motion data used to calculate a fatigue indicator for each muscle of the user can be collected. In a case where the motion is measured using the IMU, data including at least an acceleration and an angular velocity is measured as the motion data. The system for measuring the motion of the user is not limited to those exemplified here.

The ground reaction force measurement unit 122 measures a ground reaction received from a floor surface when the user walks. For example, the ground reaction force measurement unit 122 measures three component forces (vertical, lateral, and longitudinal components) of the ground reaction and a ground reaction force acting point as the ground reaction force data. The method for measuring the ground reaction force by the ground reaction force measurement unit 122 is not particularly limited. The ground reaction force measurement unit 122 outputs the ground reaction force data that is a measurement result to the measurement data transmission unit 123.

For example, if a force plate is disposed on the floor surface where the user walks, the ground reaction force can be measured. However, in that case, because it is necessary to dispose the force plate on the floor surface, it is difficult to measure the ground reaction force of the user in daily life. For example, if a pressure sensor is provided in a footwear of the user instead of the force plate, it is possible to easily measure the ground reaction force of the user in daily life. The method for measuring the ground reaction force is not limited to those exemplified here.

The measurement data transmission unit 123 acquires the motion data from the motion measurement unit 121 and acquires the ground reaction force data from the ground reaction force measurement unit 122. The measurement data transmission unit 123 generates measurement data in which the motion data and the ground reaction force data acquired for the same user are associated with a time when the data is acquired. That is, the measurement data includes at least an identifier of the user, the motion data and the ground reaction force data of the user, and the times when these pieces of data are acquired. The measurement data transmission unit 123 transmits the measurement data to the information distribution device 11 or the terminal device 13 via the network 15.

The detailed configuration of the data acquisition device 12 has been described above. The configuration in FIG. 3 is an example and does not limit the configuration of the data acquisition device 12. The data acquisition device 12 includes the motion measurement unit 121 and the ground reaction force measurement unit 122 and can be implemented as an electronic device that has a communication function capable of communicating with the information distribution device 11 or the terminal device 13. The data acquisition device 12 may have a configuration that includes the motion measurement unit 121 and the ground reaction force measurement unit 122 as different devices. The data acquisition device 12 may be configured as software or an application installed to the terminal device 13.

For example, the data acquisition device 12 can be wiredly connected to the information distribution device 11 and the terminal device 13 using a communication cable such as a Local Area network (LAN) cable or a Universal Serial Bus (USB) cable. For example, the data acquisition device 12 can be connected to the information distribution device 11 and the terminal device 13 by wireless communication such as the Bluetooth (registered trademark), a wireless LAN, or the like.

[Information Distribution Device]

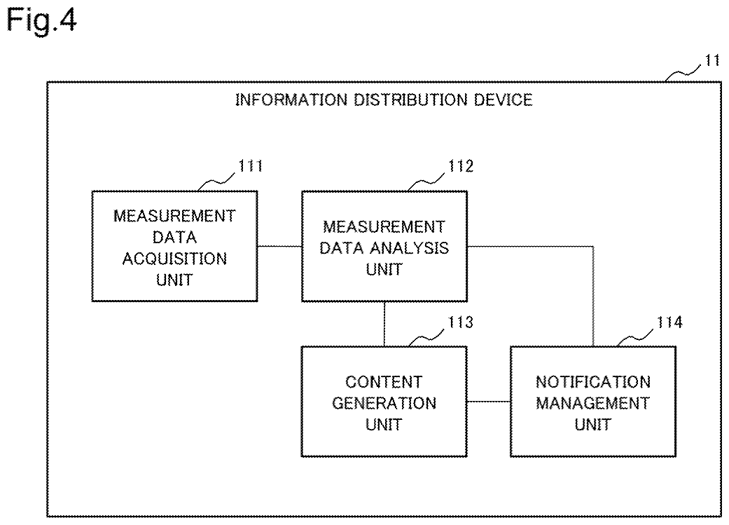

FIG. 4 is a block diagram illustrating an example of the detailed configuration of the information distribution device 11. As illustrated in FIG. 4, the information distribution device 11 includes a measurement data acquisition means 111, a measurement data analysis unit 112, a content generation unit 113, and a notification management unit 114.

The measurement data acquisition means 111 receives the measurement data transmitted from the data acquisition device 12 or the terminal device 13 via the network 15. The measurement data acquisition means 111 outputs the received measurement data to the measurement data analysis unit 112.

The measurement data analysis unit 112 acquires the measurement data from the measurement data acquisition means 111. The measurement data analysis unit 112 analyzes the motion data and the ground reaction force data included in the acquired measurement data and calculates a fatigue indicator of the user. The measurement data analysis unit 112 may be configured to estimate the ground reaction force data from the measured motion data and calculate the fatigue indicator using a measured value of the motion data and an estimated value of the ground reaction force data. In the following, in a case where the fatigue indicator is calculated using the measured value of the motion data and the estimated value of the ground reaction force data, it may be expressed that the fatigue indicator is estimated using the motion data. The measurement data analysis unit 112 outputs the calculated fatigue indicator to the content generation unit 113 and the notification management unit 114.

Here, a method for calculating the fatigue indicator by the measurement data analysis unit 112 will be described.

The measurement data analysis unit 112 analyzes the motion data and the ground reaction force data of the user using a musculo-skeletal model and calculates time-series data of a muscle activity amount. The measurement data analysis unit 112 may calculate time-series data of a muscle activity amount of a predetermined muscle or may calculate time-series data of a muscle activity amount of a plurality of muscles.

The measurement data analysis unit 112 calculates time-series data m (t) of a metabolic cost m indicating an amount of energy consumed by the muscle per unit time using the time-series data of the muscle activity amount. The metabolic cost m is related to a fatigue accumulation rate. For example, kcal/hour, cal/second, or the like is used as the unit of the metabolic cost m. The measurement data analysis unit 112 may calculate the metabolic cost m for each muscle or may calculate a total value of the metabolic costs of the respective muscles as the metabolic cost m.

The measurement data analysis unit 112 calculates a fatigue indicator F using the time-series data m (t) of the metabolic cost m. The fatigue indicator F increases as the user's fatigue increases. When the fatigue indicator F is zero, a state where the user feels no fatigue is indicated.

For example, the measurement data analysis unit 112 derives a function f (t) obtained by subtracting a recovery speed a from the time-series data m (t) of the metabolic cost m (expression 1).

ƒ(t)=m(t)−α (1)

Then, the measurement data analysis unit 112 calculates a fatigue indicator F (T) at a time T by integrating the function f (t) indicated by the expression 1 at a time 0 to that at a time T as indicated in the following expression 2 (T is positive real number).

F(T)=∫₀^{T}ƒ(t)dt (2)

The measurement data analysis unit 112 may use the following expressions 3 and 4 on the basis of a value of a fatigue indicator F (t−dt) measured at a time (t−dt) before a time t by dt at the second or subsequent measurement timings. If the expressions 3 and 4 are used, it can be prevented that the fatigue indicator F (T) falls below zero.

$$\begin{matrix}f ( t ) = \{ \begin{matrix}m ( t ) - a ( F ( t — d t ) > 0 ) \\ m ( t ) ( otherwise )\end{matrix} & ( 3 ) \\ F ( T ) = max ( {\int }_{0}^{T} f ( t ) d t , 0 ) & ( 4 )\end{matrix}$$

Although the recovery speed a fluctuates depending on environmental factors such as an age, a constitution, and a physical condition of the user, a temperature, and a humidity, the recovery speed a is preset on the basis of a general muscle fatigue recovery speed. A specific method for calculating the recovery speed a will be described later.

The content generation unit 113 acquires the fatigue indicator F from the measurement data analysis unit 112. The content generation unit 113 generates content to be distributed to a user by using the acquired fatigue indicator F. The content generation unit 113 outputs the generated content to the notification management unit 114.

For example, in a case where the fatigue indicator F is large, the content generation unit 113 generates content for recommending a user to take a break. For example, in a case where the fatigue indicator F is small, the content generation unit 113 generates content for recommending the user to resume the activity. The content generated by the content generation unit 113 is not particularly limited as long as the content is based on the fatigue indicator F. The content generated according to the fatigue indicator F will be described in the example embodiment below using specific examples.

The notification management unit 114 acquires the fatigue indicator F from the measurement data analysis unit 112 and acquires the content from the content generation unit 113. The notification management unit 114 manages a timing when the content is distributed to the user on the basis of the acquired fatigue indicator F. In other words, the notification management unit 114 manages a content push notification issuing timing on the basis of the acquired fatigue indicator F.

When determining that it is a timing for distributing the content to the user, the notification management unit 114 acquires the content from the content generation unit 113 and distributes the content to the terminal device 13 of the user. The notification management unit 114 may be configured to acquire the content generated by the content generation unit 113 each time and distribute the content that has been acquired at the time when it is determined that it is a timing for distributing the content to the terminal device 13 of the user.

For example, the notification management unit 114 monitors the time-series data of the fatigue indicator F and manages a timing for distributing the content on the basis of a relationship between the fatigue indicator F and a predetermined threshold. The predetermined threshold may be set for each user or may be uniformly the same. The timing when the notification management unit 114 distributes the content to the terminal device 13 of the user may be a time when the fatigue indicator F becomes equal to the predetermined threshold, or a time when the fatigue indicator F exceeds the predetermined threshold, or a time when the fatigue indicator F falls below the predetermined threshold.

For example, the notification management unit 114 detects the time when the fatigue indicator F of the user exceeds the predetermined threshold as a fatigue detection timing. When detecting the fatigue detection timing, the notification management unit 114 distributes the content that recommends the user to take a break to the user. For example, the notification management unit 114 detects the time when the fatigue indicator F of the user falls below the predetermined threshold as a recovery detection timing. When detecting the recovery detection timing, the notification management unit 114 distributes the content that recommends the user to resume the activity to the user. The plurality of predetermined thresholds may be set, instead of the single predetermined threshold. If the plurality of predetermined thresholds is set, it is possible to prepare the distribution of the content before the content distribution timing.

[Recovery Speed]

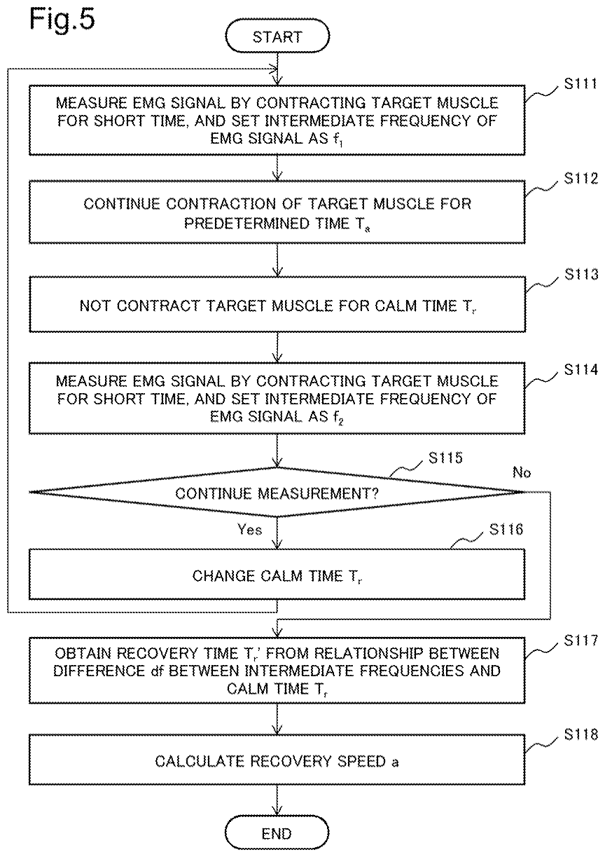

Here, a method for obtaining the recovery speed a by a muscle fatigue measurement experiment will be described with reference to the drawings. FIG. 5 is a flowchart for explaining how to obtain the recovery speed a. It is assumed that measurement according to the flowchart in FIG. 5 be performed in advance. Processing along the flowchart in FIG. 5 may be configured as a program to be executed by a computer.

A state of the muscle fatigue can be evaluated on the basis of frequency analysis of an Electromyography (EMG) signal. When the muscle is fatigued, an intermediate frequency of the EMG signal related to the muscle decreases. The intermediate frequency is a frequency that equally divides an area of a power spectrum of the EMG signal. That is, by comparing the intermediate frequencies of the EMG signal related to each muscle before and after exercise, it can be determined whether the muscle is fatigued.

First, in order to measure the EMG signal before the muscle is fatigued, the EMG signal is measured by contracting a target muscle for a short time (also referred to as first time), and an intermediate frequency of the EMG signal is set as f₁ (also referred to as first intermediate frequency) step S111). For example, in a case where the rectus femoris muscle is set as a target, an exercise for sitting on a chair and lifting the foot in the air is performed for a short time (for example, one second).

Next, in order to cause muscle fatigue, the contraction of the target muscle is continued for a predetermined time Tₐ (step S112). For example, in a case where the rectus femoris muscle is set as a target, the exercise for sitting on a chair and lifting the foot in the air is performed. In this case, for example, the predetermined time Tₐ is set to 10 seconds.

Next, in order to recover the muscle fatigue, the target muscle is not contracted for a calm time Tᵣ (also referred to as second time) (step S113). For example, in a case where the rectus femoris muscle is set as a target, the foot is placed on the ground to rest for the calm time Tᵣ.

Then, as in step S111, the EMG signal is measured by contracting a target muscle for a short time (first time), and an intermediate frequency of the EMG signal is set as f₂ (also referred to as second intermediate frequency) (step S114).

Here, in a case where the measurement is continued (Yes in step S115), the calm time Tᵣ is changed (step S116). After step S116, the procedure returns to step S111. For example, the calm time Tᵣ is set to gradually increase each time the measurement is repeated. The calm time Tᵣ may be set to gradually decrease each time the measurement is repeated. It is preferable to allow sufficient measurement intervals in such a way that the fatigue does not affect next measurement.

Steps S111 to S114 described above are assumed as one time of measurement, and the calm time Tᵣ is gradually increased in step S116, and this is repeated a plurality of times. As a result, a pair of the intermediate frequencies f₁ and f₂ is calculated for each calm time Tᵣ. The recovery speed a may be obtained by repeating the measurement while randomly changing the calm time Tᵣ.

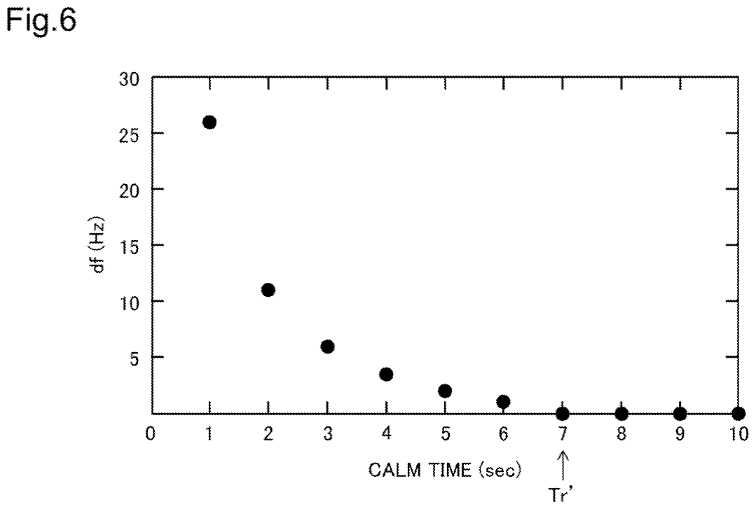

Then, a recovery time Tᵣ′ is obtained from a relationship between the calm time Tᵣ and a difference between the intermediate frequencies df (=f₁−f₂) (step S117). Specifically, as illustrated in FIG. 6, a scatter diagram is created in which the calm time Tᵣ and the difference between the intermediate frequencies df are respectively plotted on the horizontal axis and the vertical axis, and a time when the difference between the intermediate frequencies df becomes zero is obtained as the recovery time Tᵣ′. When the calm time Tᵣ is short, the muscle fatigue in step S112 remains in step S114, and accordingly, the difference between the intermediate frequencies df is a large value. On the other hand, when the calm time is longer, the muscle fatigue is recovered. Therefore, the recovery speed a approaches zero. Therefore, the calm time Tᵣ at which the difference between the intermediate frequencies df becomes zero first is related to the recovery time Tᵣ′.

The recovery time Tᵣ′ can be assumed as a period before the fatigue accumulated in the predetermined time Tₐ in which the muscle is contracted is recovered. Therefore, the relationship as in the following expression 5 is satisfied.

∫₀^{T}^{ₐ}^{+T}^{ᵣ}^{′}(m(t)−a)dt=0 (5)

Therefore, the recovery speed a is calculated by the following expression 6 (step S118).

$$a = \frac{1}{{T}_{a} + {T}_{r} '} {\int }_{0}^{T a + {Tr}^{′}} m ( t ) d t \qquad ( 6 )$$

The detailed configuration of the information distribution device 11 has been described above. The configuration in FIG. 4 is an example and does not limit the configuration of the information distribution device 11. The information distribution device 11 can be implemented by software and an application installed to a server that is constructed in the data center, the cloud, or the like, an application server, an integrated circuits for a specific application, or the like.

[Terminal Device]

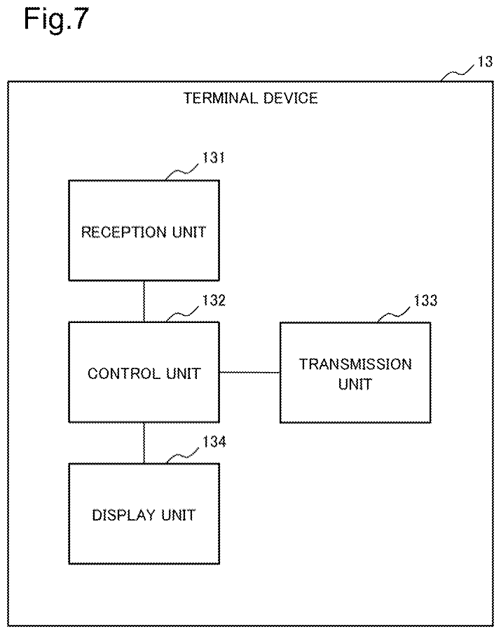

FIG. 7 is a block diagram illustrating an example of the detailed configuration of the terminal device 13. As illustrated in FIG. 7, the terminal device 13 includes a reception unit 131, a control unit 132, a transmission unit 133, and a display unit 134.

The reception unit 131 receives the measurement data from the data acquisition device 12. The reception unit 131 receives the content from the information distribution device 11. The reception unit 131 outputs the received measurement data and content to the control unit 132.

The control unit 132 acquires the measurement data from the reception unit 131. The control unit 132 outputs the acquired measurement data to the transmission unit 133 and causes the transmission unit 133 to transmit the measurement data to the information distribution device 11.

The control unit 132 acquires the content from the reception unit 131. The control unit 132 outputs the acquired content to the display unit 134 and causes the display unit 134 to display the content on a monitor (not illustrated).

The transmission unit 133 acquires the measurement data from the control unit 132. The transmission unit 133 transmits the measurement data to the information distribution device 11 according to the control of the control unit 132.

The display unit 134 acquires the content from the control unit 132. The display unit 134 displays the content on the monitor (not illustrated) according to the control of the control unit 132. Alternatively, notification of the content may be provided by voice.

The detailed configuration of the terminal device 13 has been described above. The configuration in FIG. 7 is an example and does not limit the configuration of the terminal device 13.

The configuration of the information distribution system 1 according to the present example embodiment has been described above.

The configuration of the information distribution system 1 according to the present example embodiment is not limited to the example described above, and various changes and additions may be made.

(Operation)

Next, an operation of the information distribution system 1 according to the present example embodiment will be described with reference to the drawings. Here, an operation of the information distribution device 11 included in the information distribution system 1 will be described.

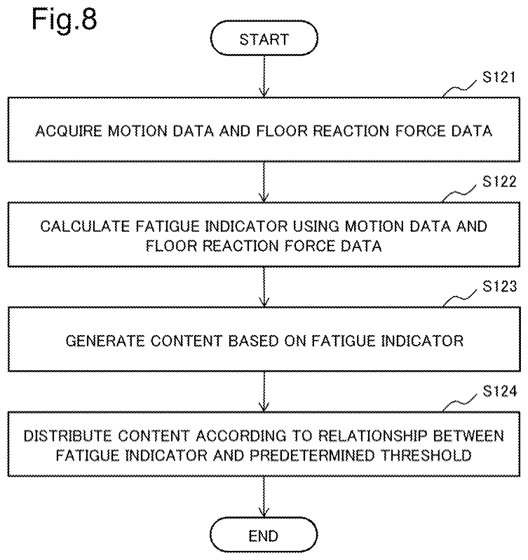

FIG. 8 is a flowchart for explaining an outline of the operation of the information distribution device 11. In the following description along the flowchart in FIG. 8, the information distribution device 11 will be described as a subject of the operation.

In FIG. 8, first, the information distribution device 11 acquires motion data and ground reaction force data of a user from the terminal device 13 (step S121).

Next, the information distribution device 11 calculates a fatigue indicator F using the acquired motion data and ground reaction force data (step S122).

Next, the information distribution device 11 generates content based on the calculated fatigue indicator F (step S123).

Then, the information distribution device 11 distributes the content according to a relationship between the fatigue indicator F and a predetermined threshold (step S124).

The outline of the operation of the information distribution device 11 according to the present example embodiment has been described above. Various changes and additions may be applied to each step in the flowchart in FIG. 8. Another step may be added between the steps in the flowchart in FIG. 8.

As described above, an information processing apparatus according to the present example embodiment acquires the measurement data regarding the motion of the user, calculates the fatigue indicator indicating the degree of the fatigue of the user by analyzing the measurement data, generates the content based on the fatigue indicator, and distributes the content on the basis of the fatigue indicator.

The information processing apparatus according to the present example embodiment includes a measurement data acquisition means, a measurement data analysis unit, a content generation unit, and a notification management unit. The measurement data acquisition means acquires the measurement data regarding the motion of the user. The measurement data analysis unit analyzes the measurement data acquired by the measurement data acquisition means and calculates the fatigue indicator indicating the degree of the fatigue of the user. The content generation unit generates the content based on the fatigue indicator calculated by the measurement data analysis unit. The notification management unit distributes the content generated by the content generation unit on the basis of the fatigue indicator.

For example, an analysis result obtained by analyzing the measurement data by the measurement data analysis unit using the musculo-skeletal model is calculated as a fatigue indicator. For example, the measurement data acquisition means acquires the motion data and the ground reaction force of the user as the measurement data, and the measurement data analysis unit calculates the fatigue indicator using the motion data and the ground reaction force. For example, the measurement data analysis unit estimates the ground reaction force using the measured value of the motion data and calculates the fatigue indicator using the measured value of the motion data and the ground reaction force estimated from the motion data.

The ground reaction force is estimated by using the measured value of the motion data, and the fatigue indicator is calculated by using the measured value of the motion data and the ground reaction force estimated from the motion data.

For example, the measurement data analysis unit calculates the time-series data of the metabolic cost indicating the amount of the energy consumed by the muscle per unit time using the time-series data of the muscle activity amount of the user. Then, the measurement data analysis unit calculates the fatigue indicator by integrating the function obtained by subtracting the recovery speed of the muscle of the user from the time-series data of the metabolic cost.

For example, the measurement data analysis unit calculates the fatigue indicator using the recovery speed obtained by comparing the intermediate frequencies of the EMG signals related to the muscle of the user before and after the exercise. More specifically, the measurement data analysis unit calculates the fatigue indicator using the recovery speed obtained from a correspondence relationship between a difference between the first intermediate frequency and the second intermediate frequency and the second time. The first intermediate frequency is an intermediate frequency of the EMG signal measured when the muscle of the user is contracted for the first time. The second intermediate frequency is an intermediate frequency of the EMG signal when the muscle of the user is contracted for a predetermined time, is relaxed for the second time, and then, contracted for the first time. The measurement data analysis unit calculates the fatigue indicator using the recovery speed that is obtained by performing measurements of the first intermediate frequency and the second intermediate frequency a plurality of times while changing the second time.

For example, the notification management unit distributes the content at any timing when the fatigue indicator exceeds a first threshold. The notification management unit distributes the content at any timing when the fatigue indicator falls below a second threshold that is smaller than the first threshold.

According to the present example embodiment, it is possible to calculate the fatigue indicator of the user using the data including the motion data and the ground reaction force of the user and distribute the content according to the fatigue degree of the user. Therefore, the user who uses this system can acquire the suitable content according to the fatigue degree of the user at an appropriate timing. As a result, according to the present example embodiment, the user can feel the convenience of the service. For example, if an appropriate stretch menu according to the fatigue degree can be provided to the user, the user can feel the convenience of the service.

According to the present example embodiment, it is possible to provide secondary information desired to be viewed by the user in association with the content at an appropriate timing. For example, if an advertisement relating to a product desired by the user is distributed together with the content according to the fatigue degree of the user, there is a possibility that an opportunity to purchase a product increases.

### Second Example Embodiment

Next, an information distribution system according to a second example embodiment of the present example embodiment will be described with reference to the drawings. The information distribution system according to the present example embodiment is different from the information distribution system according to the first example embodiment in that a database that stores content is included.

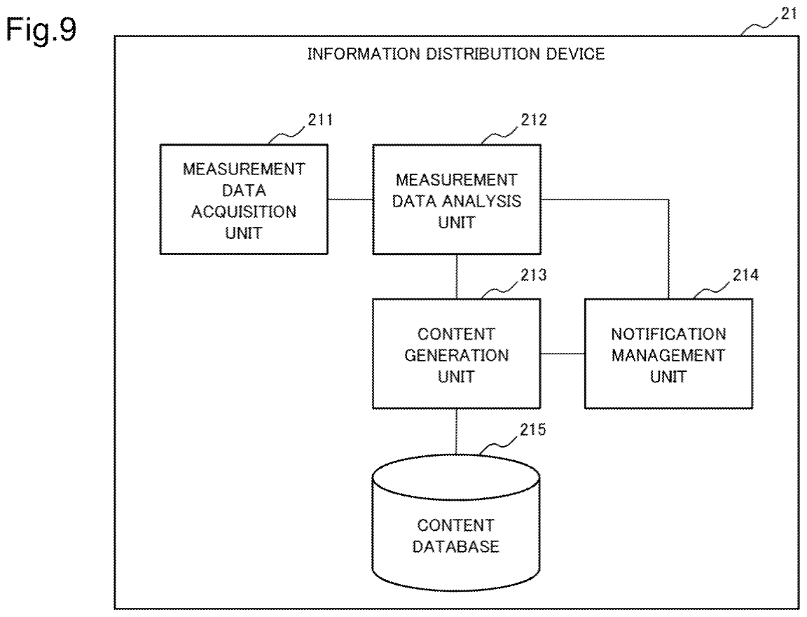

FIG. 9 is a block diagram illustrating an example of a configuration of an information distribution device 21 included in the information distribution system according to the present example embodiment. As illustrated in FIG. 9, the information distribution device 21 includes a measurement data acquisition means 211, a measurement data analysis unit 212, a content generation unit 213, a notification management unit 214, and a content database 215. Because the measurement data acquisition means 211, the measurement data analysis unit 212, the content generation unit 213, and the notification management unit 214 have similar configurations to those in the information distribution device 21 according to the first example embodiment, differences will be mainly described.

The content database 215 stores content related to a muscle of a user. For example, in the content database 215, content that is effective for fatigue recovery of each muscle is stored in association with the muscle. Here, as an example, a method will be described for generating content with reference to a table in a case where an individual metabolic cost of a predetermined muscle of a lower limb such as the gluteus maximus, the rectus femoris muscle, the medial great muscle, the semimembranosus, the biceps femoris, the soleus, the gastrocnemius muscle, the tibialis anterior muscle, or the like is calculated.

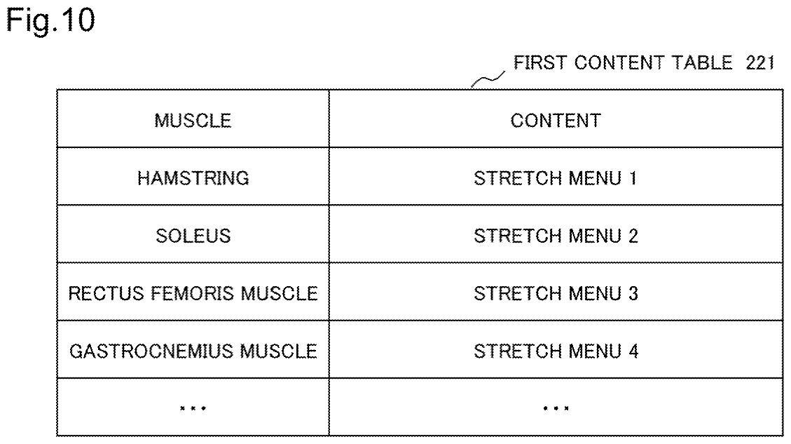

FIG. 10 is a first content table 221 that includes content that is effective for fatigue recovery of each muscle. In the first content table 221, stretch menus that are respectively effective for the hamstring, soleus, rectus femoris muscle, gastrocnemius muscle, . . . are included.

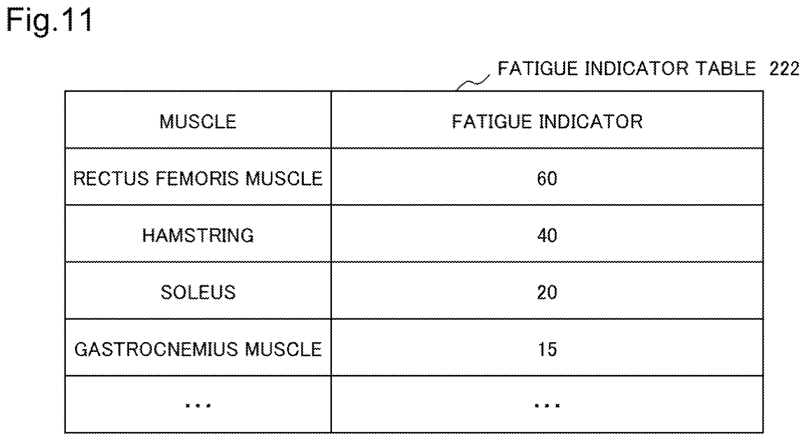

The content generation unit 213 arranges muscles in decreasing order of a fatigue indicator when acquiring a fatigue indicator F. FIG. 11 is a fatigue indicator table 222 in which a fatigue indicator for each muscle calculated from measurement data of a user is included. From the fatigue indicator table 222, it can be read that the fatigue indicators of the rectus femoris muscle, the hamstring, the soleus, and the gastrocnemius muscle are respectively 60, 40, 20, and 15.

The content generation unit 213 acquires content that is highly effective for the muscle having a larger fatigue indicator with reference to the content database 215. Because the fatigue indicator of the rectus femoris muscle is the largest in FIG. 11, the content generation unit 213 refers to the first content table 221 and selects a stretch menu 3 as content related to the rectus femoris muscle. The muscle to be a target of content generation by the content generation unit 213 is not only the muscle having the largest fatigue indicator and may be a plurality of muscles in order of the fatigue indicator from the top. The content generation unit 213 may be configured to generate a single piece of content related to a combination of the plurality of muscles. The content generation unit 213 may be configured to generate the content for all the muscles of which the fatigue indicator exceeds a predetermined threshold.

The information distribution system according to the present example embodiment has been described above.

As described above, the information distribution device according to the present example embodiment includes the content database that stores the content to be selected by the content generation unit.

For example, the content database stores the first content table that includes the content associated with at least a single muscle. The content generation unit generates the fatigue indicator table including the fatigue indicator for each muscle, refers to the first content table, and selects the content related to the muscle selected according to the fatigue indicator included in the fatigue indicator table.

A fatigued part and a degree of fatigue of the user vary depending on an individual and a behavior in the day, or a habit. Because a general information distribution service does not have a mechanism for generating content according to the fatigued part of the user, it is not necessarily possible to distribute content suitable for the user.

On the other hand, the information distribution system according to the present example embodiment can generate highly effective content according to the fatigue degree of the user and can distribute the generated content at a suitable timing. For example, in this system, information such as “Stretch your calves”, “Massage your thighs”, or the like is displayed on the terminal device of the user according to the muscle that is estimated to be tired. Therefore, the user who receives the provision of the information distribution service of this system can acquire content suitable for relieving the fatigue at an appropriate timing when the user feels fatigue while going out. That is, according to the present example embodiment, because the content can be set in detail for each muscle, it is possible to distribute the content more suitable for the user than the first example embodiment.

### Third Example Embodiment

Next, an information distribution system according to a third example embodiment of the present example embodiment will be described with reference to the drawings. The information distribution system according to the present example embodiment is different from those in the first and second example embodiments in that position information of the terminal device is used. In the following, the information distribution device and the data acquisition device are assumed to be similar to those in the second example embodiment, and description of the overlapped portions will be omitted.

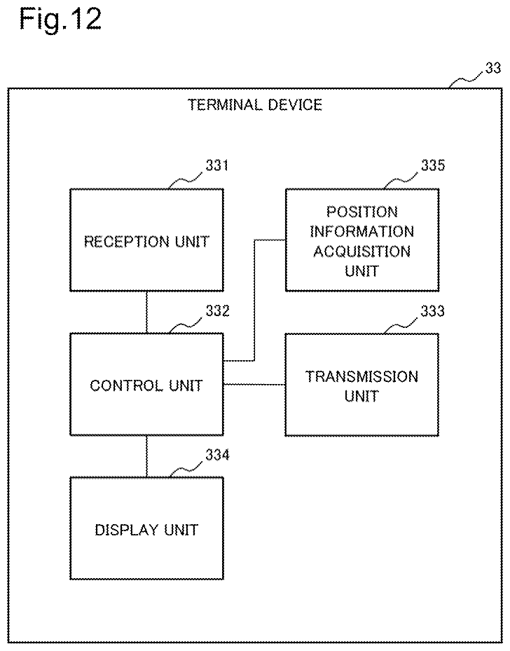

FIG. 12 is a block diagram for explaining an example of a configuration of a terminal device 33 included in the information distribution system according to the present example embodiment. As illustrated in FIG. 12, the terminal device 33 includes a reception unit 331, a control unit 332, a transmission unit 333, a display unit 334, and a position information acquisition unit 335. The reception unit 331, the control unit 332, the transmission unit 333, and the display unit 334 have configurations similar to those in the terminal device 13 according to the first example embodiment, differences will be mainly descried.

The position information acquisition unit 335 acquires position information of the terminal device 33. The position information acquisition unit 335 outputs the acquired position information to the control unit 332.

For example, the position information acquisition unit 335 can be implemented as a Global Positioning System (GPS) for performing positioning on the basis of signals from a plurality of satellites. The position information acquisition unit 335 may acquire the position information from information included in radio waves received by the reception unit 331 of the terminal device 33 in addition to the GPS.

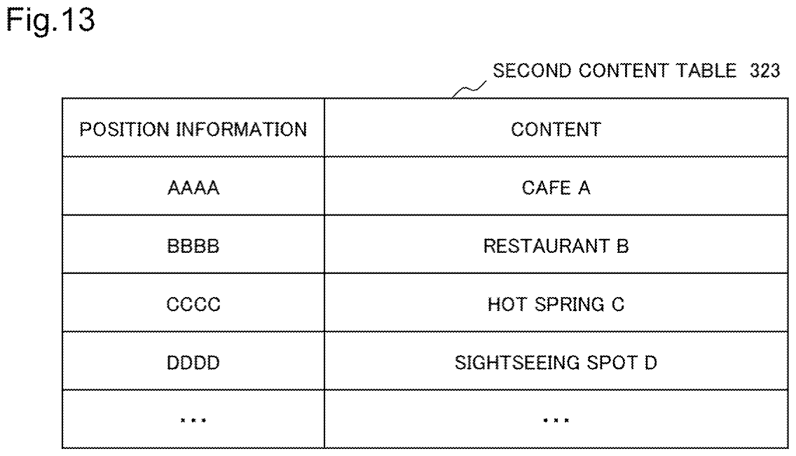

FIG. 13 is an example of a second content table 323 including content related to the position information. In the second content table 323 in FIG. 13, position information AAAA is related to a cafe A, position information BBBB is related to a restaurant B, position information CCCC is related to a hot spring C, and position information DDD is related to a sightseeing spot D.

The control unit 332 acquires the position information from the position information acquisition unit 335. The control unit 332 outputs the measurement data acquired from the reception unit together with position information related to the measurement data to the transmission unit 333.

The transmission unit 333 acquires the position information in addition to the measurement data from the control unit 332. The transmission unit 333 transmits the measurement data and the position information to the information distribution device 11 according to the control of the control unit 332.

For example, an information distribution device that receives the measurement data and the position information distributes content related to the position information to the terminal device 33 at a time when the terminal device 33 reaches a predetermined distance from a position indicated by the position information. A user of the terminal device 33 can acquire suitable information by confirming the content.

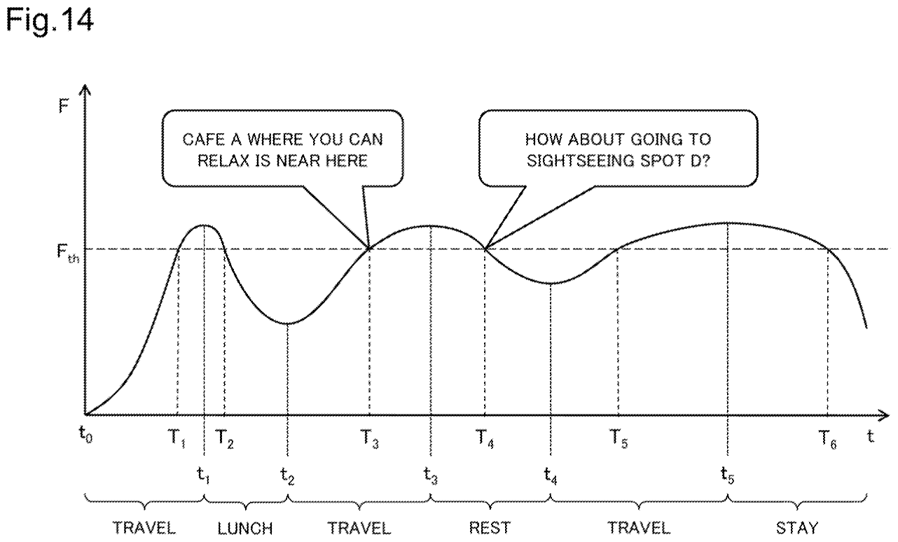

FIG. 14 is a graph for explaining an example for distributing content suitable for a user on a trip. In FIG. 14, the horizontal axis indicates time t, and the vertical axis indicates a fatigue indicator F. In the example in FIG. 14, when the fatigue indicator exceeds a predetermined threshold Fₜₕ, in a case where there is content that is distributed in relation with the position information of the terminal device 33, it is assumed that the content be distributed. In the example in FIG. 14, the fatigue indicator F exceeds the predetermined threshold Fₜₕ at times T₁, T₃, and T₅, and the fatigue indicator F falls below the predetermined threshold Fₜₕ at times T₂, T₄, and T₆. Then, the fatigue indicator F is maximized at times t₁, t₃, and t₅, and the fatigue indicator F is minimized at times t₂ and t₄. The fatigue indicator F increases in a period when the user travels and decreases in a period when the user does not travel (lunch, rest, stay).

In the example in FIG. 14, it is assumed that the terminal device 33 be positioned within a range in which the cafe A and the sightseeing spot D are distributed as the content. When the fatigue indicator F exceeds the predetermined threshold Fₜₕ, at the time T₃, the information distribution device distributes the cafe A to the terminal device 33 as the content. The terminal device 33 that has received the content displays, for example, “The cafe A where you can relax is near here” on a monitor (not illustrated). When the fatigue indicator F falls below the predetermined threshold Fₜₕ at the time T₄, the information distribution device distributes the sightseeing spot D to the terminal device 33 as the content. The terminal device 33 that has received the content displays, for example, “How about going to the sightseeing spot D?” on the monitor (not illustrated). The user of the terminal device 33 obtains a guideline of the next action by confirming the content displayed on the monitor (not illustrated) of the terminal device 33.

The configuration of the information distribution system according to the present example embodiment has been described above. The information distribution system according to the present example embodiment may be configured to distribute the content based on the fatigue indicator indicated in the second example embodiment, in addition to the content related to the position information. The information distribution system according to the present example embodiment may be configured to distribute the content related to the position information in addition to the content based on the fatigue indicator indicated in the second example embodiment.

(Operation)

Next, an operation of the information distribution system according to the present example embodiment will be described. Here, an operation of the information distribution device (not illustrated) included in the information distribution system will be described.

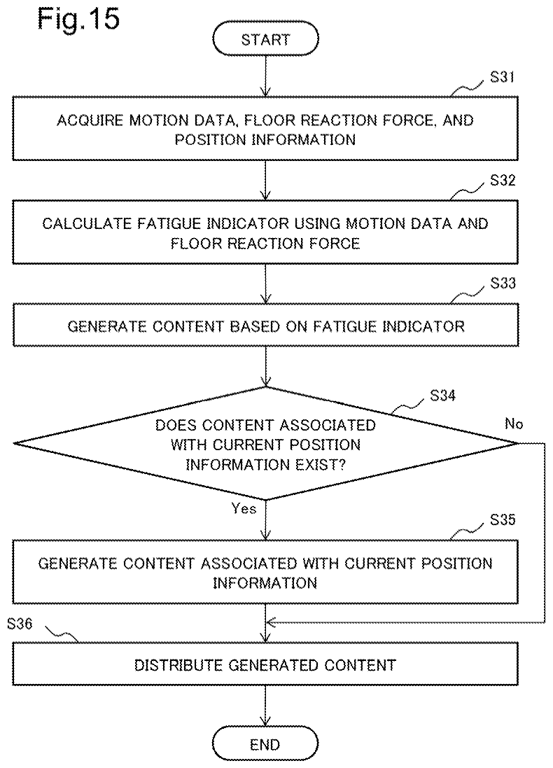

FIG. 15 is a flowchart for explaining an outline of the operation of the information distribution device. In the following description along the flowchart in FIG. 15, the information distribution device will be described as a subject of the operation. In the example in FIG. 15, an example will be described in which the content is generated on the basis of the fatigue indicator F and the content is generated according the position information of the terminal device 33.

In FIG. 15, first, the information distribution device acquires motion data, ground reaction force data, and position information of a user from the terminal device 33 (step S31).

Next, the information distribution device calculates the fatigue indicator F using the acquired motion data and ground reaction force data (step S32).

Next, the information distribution device generates the content based on the calculated fatigue indicator F (step S33).

Here, the information distribution device confirms whether content associated with current position information of the terminal device 33 exists (step S34).

In a case where the content associated with the current position information of the terminal device 33 exists (Yes in step S34), the information distribution device generates the content associated with the current position information of the terminal device 33 (step S35). On the other hand, in a case where the content associated with the current position information of the terminal device 33 does not exist (No in step S34), the procedure proceeds to step S36.

Then, the information distribution device distributes the generated content (step S36). In a case of Yes in step S34, the information distribution device distributes the content based on the fatigue indicator and the content associated with the current position information. On the other hand, in a case of No in step S34, the information distribution device distributes the content based on the fatigue indicator.

The outline of the operation of the information distribution device according to the present example embodiment has been described above. Various changes and additions may be applied to each step in the flowchart in FIG. 15. Another step may be added between the steps in the flowchart in FIG. 15.

As described above, the content database of the information distribution device according to the present example embodiment stores the second content table including the content associated with the position information. The content generation unit of the information distribution device according to the present example embodiment acquires the position information of the terminal device used by the user and selects the content related to the position information of the terminal device by referring to the second content table. The notification management unit according to the present example embodiment distributes the content related to the position information of the terminal device on the basis of the fatigue indicator.

According to the present example embodiment, because it is possible to provide the content in consideration of the position information of the user to the user, it is possible to distribute content more suitable for the user than the first and second example embodiments.

Generally, degrees of accumulation and recovery of fatigue differ according to the user. Therefore, because a general information distribution service does not have a mechanism for generating content according to the degrees of the accumulation and the recovery of the fatigue of the user, it is not necessarily possible to distribute the content suitable for the user at an appropriate timing.

On the other hand, the information distribution system according to the present example embodiment detects fatigue before the user becomes aware of the fatigue depending on the degree of the accumulation of the fatigue of the user and can distribute the content suitable for the user at an appropriate timing. For example, according to the present example embodiment, in a state where the user on a trip is fatigued near a cafe, information such as “There is a cafe near here where you can relax” can be distributed. The information distribution system according to the present example embodiment can distribute the content suitable for the user at an appropriate timing according to the degree of the recovery of the fatigue of the user. For example, at a timing when the user takes a break for about one hour and the fatigue is recovered, information such as “How about going to a nearby sightseeing spot?” can be distributed.

### Fourth Example Embodiment

Next, an information distribution system according to a fourth example embodiment of the present example embodiment will be described with reference to the drawings. The information distribution system according to the present example embodiment is different from those of the first to third example embodiments in that content associated with time is generated. In the following, the information distribution device and the data acquisition device are assumed to be similar to those in the second example embodiment, and description of the overlapped portions will be omitted.

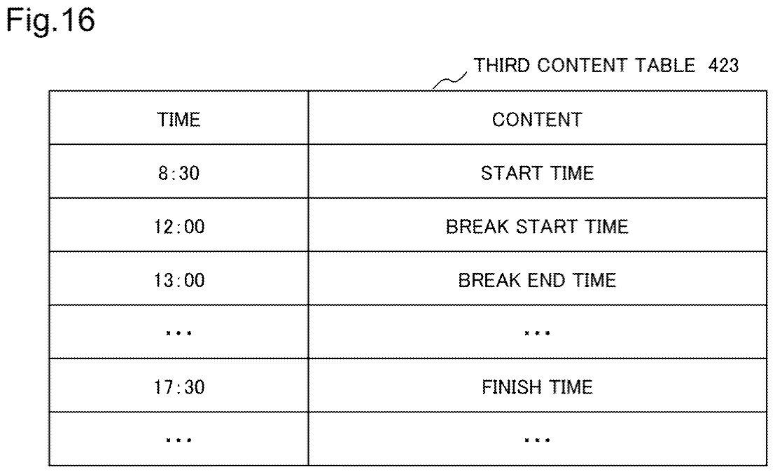

FIG. 16 is an example of a third content table 423 including content related to time. The third content table 423 in FIG. 16 is an example of content that is provided in a place where a predetermined action is required at a specific time such as a workplace, a school, or a hospital. In the third content table 423 in FIG. 16, 8:30 is set as a start time, 12:00 is set as a break start time, 13:00 is set as a break end time, . . . , and 17:30 is set as a finish time.

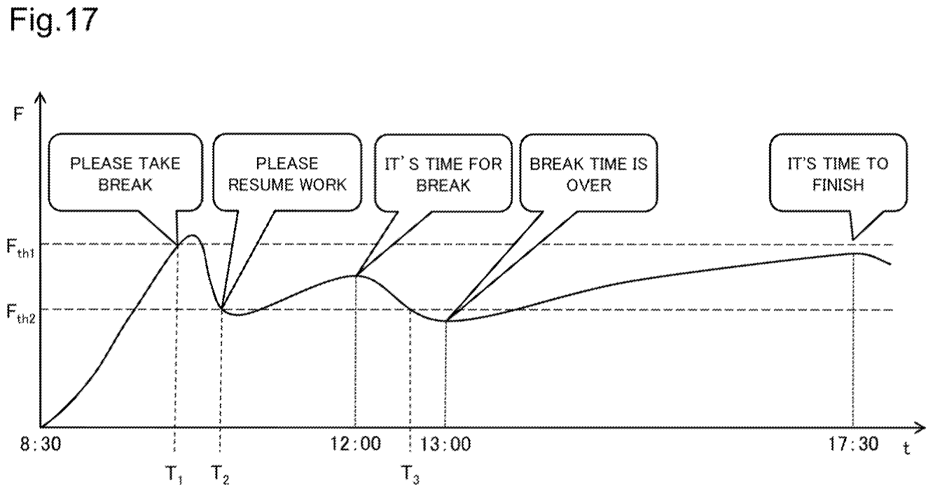

FIG. 17 is a graph for explaining an example in which the content related to the time is distributed in addition to the content according to the fatigue indicator of the user. In FIG. 17, the horizontal axis indicates time t, and the vertical axis indicates the fatigue indicator F. In the example in FIG. 17, it is assumed that content based on the accumulation of the fatigue of the user be distributed when the fatigue indicator F exceeds a first threshold Fₜₕ₁ and content based on the recovery of the fatigue of the user be distributed when the fatigue indicator F falls below a second threshold Fₜₕ₂. In the example in FIG. 17, the fatigue indicator F exceeds the first threshold Fₜₕ₁ at time T₁ and falls below the second threshold Fₜₕ₂ at times T₂ and T₃. It is assumed that the fatigue indicator F increase in a user's working period and decrease in a rest period.

In the example in FIG. 17, when the fatigue indicator F exceeds the first threshold Fₜₕ₁ at the time T₁, the information distribution device distributes content for recommending a user to have a break to a terminal device. The terminal device that has received the content displays, for example, “Please take a break” on a monitor (not illustrated). Then, when the fatigue indicator F of the user falls below the second threshold Fₜₕ₂ on the basis of the recovery speed of the user, the information distribution device distributes content for recommending the user to resume the work to the terminal device. The terminal device that has received the content displays, for example, “Please resume the work” on the monitor (not illustrated).

At 12:00, the information distribution device distributes content that notifies that a break time has come to the terminal device. The terminal device that has received the content displays, for example, “It's time for a break” on the monitor (not illustrated). Then, at 13:00, the information distribution device distributes content that notifies that the break time ends to the terminal device. The terminal device that has received the content displays, for example, “The break time is over” on the monitor (not illustrated). In a case where the fatigue indicator F falls below the second threshold Fₜₕ₂ in a period from 12:00 to 13:00 during the break time, it is not necessary to distribute content or some content may be distributed.

The configuration of the information distribution system according to the present example embodiment has been described above. The information distribution system according to the present example embodiment may be configured to distribute the content based on the position information indicated in the third example embodiment in addition to the content related to the time.

(Operation)

Next, an operation of the information distribution system according to the present example embodiment will be described. Here, an operation of the information distribution device (not illustrated) included in the information distribution system will be described.

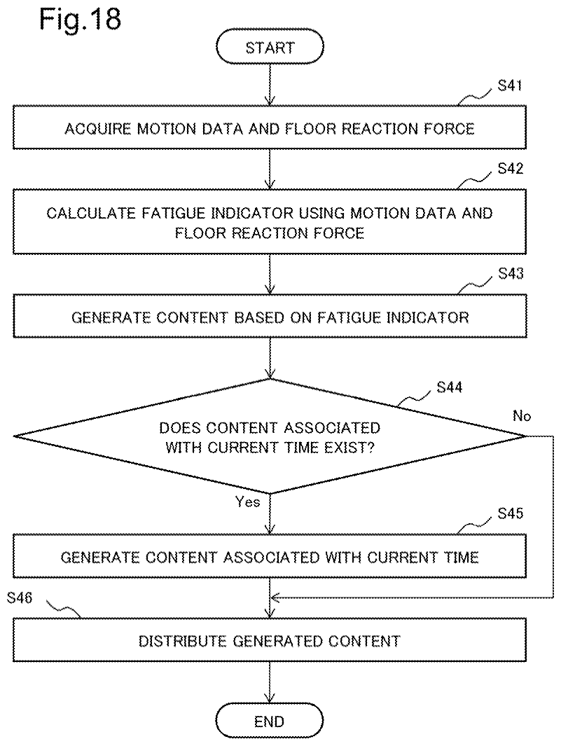

FIG. 18 is a flowchart for explaining an outline of the operation of the information distribution device. In the following description along the flowchart in FIG. 18, the information distribution device will be described as a subject of the operation. In the example in FIG. 18, an example will be described in which the content is generated on the basis of the fatigue indicator F and the content is generated according to the time.

In FIG. 18, first, the information distribution device acquires motion data and ground reaction force data of a user from the terminal device 33 (step S41).

Next, the information distribution device calculates the fatigue indicator F using the acquired motion data and ground reaction force data (step S42).

Next, the information distribution device generates the content based on the calculated fatigue indicator F (step S43).

Here, the information distribution device confirms whether content associated with current time exists (step S44).

In a case where the content associated with the current time exists (Yes in step S44), the information distribution device generates content associated with the current time (step S45). On the other hand, in a case where the content associated with the current time does not exist (No in step S44), the procedure proceeds to step S46.

Then, the information distribution device distributes the generated content (step S46). In a case of Yes in step S44, the information distribution device distributes the content based on the fatigue indicator and the content associated with the current time. On the other hand, in a case of No in step S44, the information distribution device distributes the content based on the fatigue indicator.

The outline of the operation of the information distribution device according to the present example embodiment has been described above. Various changes and additions may be applied to each step in the flowchart in FIG. 18. Another step may be added between the steps in the flowchart in FIG. 18.

As described above, the content database of the information distribution device according to the present example embodiment stores the third content table including the content associated with the time. The content generation unit of the information distribution device according to the present example embodiment selects the content related to the time by referring to the third content table. The notification management unit of the information distribution device according to the present example embodiment distributes the content related to the time on the basis of the fatigue indicator.

According to the present example embodiment, the content in consideration of the time can be provided to the user. Therefore, it is possible to distribute content more suitable for the user at a more appropriate timing than the first and second example embodiments. According to the present example embodiment, it is possible to distribute the content according to the degree of the recovery of the fatigue. Therefore, it is possible to distribute the content for recommending resume of work with respect to a user who does not resume the work even though the fatigue is recovered.

(Hardware)

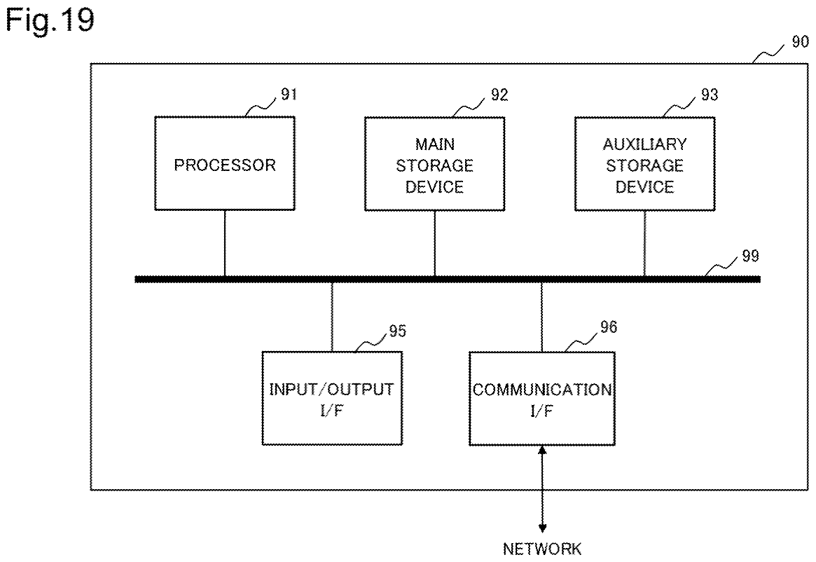

Here, a hardware configuration for executing processing of the information distribution device according to each example embodiment of the disclosure will be described using an information processing apparatus 90 in FIG. 19 as an example. The information processing apparatus 90 in FIG. 19 is an exemplary configuration that executes the processing by the information distribution device according to each example embodiment and does not limit the scope of the disclosure.

As illustrated in FIG. 19, the information processing apparatus 90 includes a processor 91, a main storage device 92, an auxiliary storage device 93, an input/output interface 95, and a communication interface 96. In FIG. 19, an interface is abbreviated and expressed as an I/F (Interface). The processor 91, the main storage device 92, the auxiliary storage device 93, the input/output interface 95, and the communication interface 96 are connected to each other via a bus 99 in such a way as to perform data communication. The processor 91, the main storage device 92, the auxiliary storage device 93, and the input/output interface 95 are connected to a network such as the Internet or an intranet via the communication interface 96.

The processor 91 develops a program stored in the auxiliary storage device 93 and the like to the main storage device 92 and executes the developed program. In the present example embodiment, it is sufficient to use a configuration using a software program installed in the information processing apparatus 90. The processor 91 executes processing by the information distribution device according to the present example embodiment.

The main storage device 92 has a region where the program is developed. It is only required for the main storage device 92 to be, for example, a volatile memory such as a Dynamic Random Access Memory (DRAM). A nonvolatile memory such as a Magnetoresistive Random Access Memory (MRAM) may be configured and added as the main storage device 92.

The auxiliary storage device 93 stores various data. The auxiliary storage device 93 includes a hard disk and a local disk such as a flash memory. It is possible that the main storage device 92 stores various data and the auxiliary storage device 93 is omitted.

The input/output interface 95 is an interface that connects the information processing apparatus 90 to peripheral devices. The communication interface 96 is an interface that connects to an external system or device through the network such as the Internet or the intranet on the basis of standards and specifications. The input/output interface 95 and the communication interface 96 may be commonly used as an interface that connects to an external device.

The information processing apparatus 90 may be configured to be connected to an input device such as a keyboard, a mouse, a touch panel, or the like as necessary. These input devices are used to input information and settings. In a case where the touch panel is used as the input device, it is sufficient to use a configuration in which a display screen of a display device also serves as the interface of the input device. It is sufficient that data communication between the processor 91 and the input device be relayed by the input/output interface 95.

The information processing apparatus 90 may include a display device used to display information. In a case where the display device is included, it is preferable that the information processing apparatus 90 include a display control device (not illustrated) that controls the display of the display device. It is sufficient that the display device be connected to the information processing apparatus 90 via the input/output interface 95.

The information processing apparatus 90 may include a disk drive as necessary. The disk drive is connected to the bus 99. Between the processor 91 and a recording medium (program recording medium) which is not illustrated, the disk drive relays reading of data and programs from the recording medium, writing of a processing result of the information processing apparatus 90 to the recording medium, or the like. The recording medium can be implemented, for example, by an optical recording medium such as a Compact Disc (CD) or a Digital Versatile Disc (DVD). The recording medium may be implemented by a semiconductor recording medium such as a Universal Serial Bus (USB) memory or a Secure Digital (SD) card, a magnetic recording medium such as a flexible disk, or other recording medium.

The above is an example of the hardware configuration that enables the information distribution device according to each example embodiment of the disclosure. The hardware configuration in FIG. 19 is an example of the hardware configuration that executes arithmetic processing by the information distribution device according to each example embodiment and does not limit the scope of the disclosure. A program that causes a computer to execute processing regarding the information distribution device according to each example embodiment is included in the scope of the disclosure. Moreover, a program recording medium that has recorded the program according to each example embodiment is included in the scope of the disclosure.

The components of the information distribution device according to each example embodiment can be optionally combined. The components of the information distribution device according to each example embodiment may be implemented by software or a circuit.

While the disclosure has been particularly shown and described with reference to exemplary embodiments thereof, the invention is not limited to these embodiments. It will be understood by those of ordinary skill in the art that various changes in form and details may be made therein without departing from the spirit and scope of the disclosure as defined by the claims.

Although a part or all of the example embodiments may be described as supplementary notes below, a part or all of the example embodiments are not limited to the following supplementary notes.

(Supplementary Note 1)

An information distribution device including:
a measurement data acquisition means configured to acquire measurement data regarding a motion of a user;
a measurement data analysis means configured to analyze the measurement data acquired by the measurement data acquisition means and calculate a fatigue indicator that indicates a degree of fatigue of the user;
a content generation means configured to generate content based on the fatigue indicator calculated by the measurement data analysis means; and
a notification management means configured to distribute the content generated by the content generation means based on the fatigue indicator.

(Supplementary Note 2)

The information distribution device according to supplementary note 1, in which
the measurement data analysis means
calculates an analysis result obtained by analyzing the measurement data by a musculo-skeletal model as the fatigue indicator.

(Supplementary Note 3)

The information distribution device according to supplementary note 1 or 2, in which
the measurement data acquisition means
acquires motion data and a ground reaction force of the user as the measurement data, and
the measurement data analysis means
calculates the fatigue indicator using the motion data and the ground reaction force.

(Supplementary Note 4)

The information distribution device according to supplementary note 3, in which
the measurement data analysis means
estimates the ground reaction force using the motion data and calculates the fatigue indicator using a measured value of the motion data and the ground reaction force estimated from the motion data.

(Supplementary Note 5)

The information distribution device according to any one of supplementary notes 1 to 4, in which
the measurement data analysis means
calculates time-series data of a metabolic cost that indicates an amount of energy consumed by a muscle per unit time by using time-series data of a muscle activity amount of the user and calculates the fatigue indicator by integrating a function obtained by subtracting a recovery speed of the muscle of the user from the time-series data of the metabolic cost.

(Supplementary Note 6)

The information distribution device according to supplementary note 5, in which
the measurement data analysis means
calculates the fatigue indicator using the recovery speed obtained by comparing intermediate frequencies of EMG signals related to the muscle of the user before and after an exercise.

(Supplementary Note 7)

The information distribution device according to supplementary note 5, in which
measurement of a first intermediate frequency of the EMG signal measured when a muscle of the user is contracted for a first time and measurement of a second intermediate frequency of the EMG signal when the muscle of the user is contracted for a predetermined time, is relaxed for a second time, and then contracted for the first time are performed a plurality of times while changing the second time, and the fatigue indicator is calculated using the recovery speed obtained from a correspondence relationship between a difference between the first intermediate frequency and the second intermediate frequency and the second time.

(Supplementary Note 8)

The information distribution device according to any one of supplementary notes 1 to 7, in which
the notification management means
distributes the content at any timing when the fatigue indicator exceeds a first threshold.

(Supplementary Note 9)

The information distribution device according to supplementary note 8, in which
the notification management means
distributes the content at any timing when the fatigue indicator falls below a second threshold smaller than the first threshold.

(Supplementary Note 10)

The information distribution device according to any one of supplementary notes 1 to 9, further including:
a content database configured to store the content selected by the content generation means.

(Supplementary Note 11)

The information distribution device according to supplementary note 10, in which
the content database
stores a first content table that includes the content associated with at least one muscle, and
the content generation means
generates a fatigue indicator table that includes the fatigue indicator for each muscle and selects the content related to a muscle selected according to the fatigue indicator included in the fatigue indicator table by referring to the first content table.

(Supplementary Note 12)

The information distribution device according to supplementary note 10 or 11, in which
the content database
stores a second content table that includes the content associated with position information, and
the content generation means
acquires position information of a terminal device used by the user and selects the content related to the position information of the terminal device by referring to the second content table.

(Supplementary Note 13)

The information distribution device according to any one of supplementary notes 10 to 12, in which
the content database
stores a third content table that includes the content associated with time, and
the content generation means
selects the content related to time by referring to the third content table.

(Supplementary Note 14)

An information distribution system including:
the information distribution device according to any one of supplementary notes 1 to 13; and
a data acquisition device configured to acquire the measurement data.

(Supplementary Note 15)

The information distribution system according to supplementary note 14, further including:
at least one terminal device data-communicably connected to the information distribution device and the data acquisition device.

(Supplementary Note 16)

An information distribution method including:
by an information processing server
acquiring measurement data regarding a motion of a user;
analyzing the acquired measurement data and calculating a fatigue indicator that indicates a degree of fatigue of the user;
generating content based on the calculated fatigue indicator; and
distributing the generated content based on the fatigue indicator.

(Supplementary Note 17)

A program for causing a computer to execute processing including:
processing for acquiring measurement data regarding a motion of a user;
processing for analyzing the acquired measurement data and calculating a fatigue indicator that indicates a degree of fatigue of the user;
processing for generating content based on the calculated fatigue indicator; and
processing for distributing the generated content based on the fatigue indicator.

### REFERENCE SIGNS LIST

1 information distribution system
11 information distribution device
12 data acquisition device
13 terminal device
15 network
111 measurement data acquisition means
112 measurement data analysis unit
113 content generation unit
114 notification management unit
121 motion measurement unit
122 ground reaction force measurement unit
123 measurement data transmission unit
131 reception unit
132 control unit
133 transmission unit
134 display unit
211 measurement data acquisition means
212 measurement data analysis unit
213 content generation unit
214 notification management unit
215 content database
221 first content table
222 fatigue indicator table
331 reception unit
332 control unit
333 transmission unit
334 display unit
335 position information acquisition unit
323 second content table
423 third content table

What is claimed is:

1. An information distribution device comprising:
a memory storing instructions; and
at least one processor configured to run the instructions to perform:
acquiring measurement data regarding a motion of a user;
analyzing the measurement data acquired by the measurement data acquisition means and calculating a fatigue indicator that indicates a degree of fatigue of the user;
generating content based on the fatigue indicator; and
distributing the content generated by the content generation means based on the fatigue indicator.

2. The information distribution device according to claim 1, wherein
the at least one processor is configured to run the instructions to perform:
calculating an analysis result obtained by analyzing the measurement data by a musculo-skeletal model as the fatigue indicator.

3. The information distribution device according to claim 1, wherein
the at least one processor is configured to run the instructions to perform:
acquiring motion data and a ground reaction force of the user as the measurement data, and
calculating the fatigue indicator using the motion data and the ground reaction force.

4. The information distribution device according to claim 3, wherein
the at least one processor is configured to run the instructions to perform:
estimating the ground reaction force using a measured value of the motion data and calculates the fatigue indicator using the measured value of the motion data and the ground reaction force estimated from the motion data.

5. The information distribution device according to claim 1, wherein
the at least one processor is configured to run the instructions to perform:
calculating time-series data of a metabolic cost that indicates an amount of energy consumed by a muscle per unit time by using time-series data of a muscle activity amount of the user and calculates the fatigue indicator by integrating a function obtained by subtracting a recovery speed of the muscle of the user from the time-series data of the metabolic cost.

6. The information distribution device according to claim 5, wherein
the at least one processor is configured to run the instructions to perform:
calculating the fatigue indicator using the recovery speed obtained by comparing intermediate frequencies of EMG signals related to the muscle of the user before and after an exercise.

7. The information distribution device according to claim 5, wherein
the at least one processor is configured to run the instructions to perform:
performing measurement of a first intermediate frequency of the EMG signal measured when a muscle of the user is contracted for a first time and measurement of a second intermediate frequency of the EMG signal when the muscle of the user is contracted for a predetermined time, is relaxed for a second time, and then contracted for the first time a plurality of times while changing the second time, and calculates the fatigue indicator using the recovery speed obtained from a correspondence relationship between a difference between the first intermediate frequency and the second intermediate frequency and the second time.

8. The information distribution device according to claim 1, wherein
the at least one processor is configured to run the instructions to perform:
distributing the content at any timing when the fatigue indicator exceeds a first threshold.

9. The information distribution device according to claim 8, wherein
the at least one processor is configured to run the instructions to perform:
distributing the content at any timing when the fatigue indicator falls below a second threshold smaller than the first threshold.

10. The information distribution device according to claim 1, further comprising:
a content database configured to store the content.

11. The information distribution device according to claim 10, wherein
the content database
stores a first content table that includes the content associated with at least one muscle, and
the at least one processor is configured to run the instructions to perform:
generating a fatigue indicator table that includes the fatigue indicator for each muscle and selects the content related to a muscle selected according to the fatigue indicator included in the fatigue indicator table by referring to the first content table.

12. The information distribution device according to claim 10, wherein
the content database
stores a second content table that includes the content associated with position information, and
the at least one processor is configured to run the instructions to perform:
acquiring position information of a terminal device used by the user and selects the content related to the position information of the terminal device by referring to the second content table.

13. The information distribution device according to claim 10, wherein
the content database
stores a third content table that includes the content associated with time, and
the at least one processor is configured to run the instructions to perform:
selecting the content related to time by referring to the third content table.

14. An information distribution system comprising:
the information distribution device according to claim 1; and
a data acquisition device configured to acquire the measurement data.

15. The information distribution system according to claim 14, further comprising:
at least one terminal device data-communicably connected to the information distribution device and the data acquisition device.

16. An information distribution method including:
by an information processing server
acquiring measurement data regarding a motion of a user;
analyzing the acquired measurement data and calculating a fatigue indicator that indicates a degree of fatigue of the user;
generating content based on the calculated fatigue indicator; and
distributing the generated content based on the fatigue indicator.

17. A non-transitory program recording medium that records a program for causing a computer to execute processing comprising:
processing for acquiring measurement data regarding a motion of a user;
processing for analyzing the acquired measurement data and calculating a fatigue indicator that indicates a degree of fatigue of the user;
processing for generating content based on the calculated fatigue indicator; and
processing for distributing the generated content based on the fatigue indicator.
